(19) United States
(12) Patent Application Publication
(10) Pub. No.: US 2003/0133987 A1
(43) Pub. Date: Jul. 17, 2003

(54) Drug nanoparticles from template emulsions

(76) Inventors: Sonke Svenson, Midland, MI (US); Chris J. Tucker, Midland, MI (US); Steve Lubetkin, Zionsville, IN (US); Jonathan C. Evans, Midland, MI (US); Steve S. Rosenberg, Midland, MI (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/340,079

(22) Filed: Jan. 10, 2003

Related U.S. Application Data
(60) Provisional application No. 60/348,959, filed on Jan. 14, 2002.

(51) Int. Cl. A61K 9/14; A61K 9/16; A61K 9/50

(52) U.S. Cl. 424/490; 264/4.1

(57) ABSTRACT

A method for producing micron-sized or submicron-sized drugs is disclosed, which comprises preparing a template emulsion comprising water and a templating agent; preparing a drug-containing mixture comprising a drug substance; and combining the template emulsion with the drug-containing mixture to form a template emulsion loaded with drug particles. Drug particles prepared from this process are also disclosed. The resulting drug particles demonstrate faster dissolution rates and enhanced bioavailability as compared to unprocessed drug particles and particles prepared using other processes.

### CROSS-REFERENCE TO PRIOR APPLICATION

This application claims the benefit of U.S. Provisional Application No. 60/348,959 filed Jan. 14, 2002.

### FIELD OF THE INVENTION

The present invention relates to the preparation of aqueous emulsions, and in particular to the preparation of such emulsions containing poorly water soluble pharmaceutical products or drugs.

### BACKGROUND OF THE INVENTION

High bioavailability and short dissolution times are desirable attributes of a pharmaceutical end product. Bioavailability is a term meaning the degree to which a pharmaceutical product, or drug, becomes available to the target tissue after being administered to the body. Poor bioavailability is a significant problem encountered in the development of pharmaceutical compositions, particularly those containing an active ingredient that is poorly soluble in water. For example, upon oral administration poorly water soluble drugs tend to be eliminated from the gastrointestinal tract before being absorbed into the circulation.

It is known that the rate of dissolution of a particulate drug can increase with increasing surface area, i.e., decreasing particle size. Consequently, efforts have been made to control the size and size range of drug particles in pharmaceutical compositions. For example, wet milling techniques have been used, as described in U.S. Pat. No. 5,145,684. However, such wet milling techniques exhibit problems associated with contamination from the grinding media. Moreover, exposing a drug substance to excessive mechanical shear or exceedingly high temperatures can cause the drug to change or lose activity due to decomposition of the active compound, or due to recrystallyzation processes, i.e., formation of different crystalline polymorphs or transformation, at least in part, from the crystalline to the amorphous state, as described by Florence et al, Effect of Particle Size Reduction on Digoxin Crystal Properties, Journal of Pharmaceutics and Pharmacology, Vol. 26, No. 6, 479-480 (1974), and R. Suryanarayanan and A. G. Mitchell, Evaluation of Two Concepts of Crystallinity Using Calcium Gluceptate as a Model Compound, International Journal of Pharmaceutics, Vol. 24, 1-17 (1985). In addition, wet milling techniques always result in the presence of a fraction of larger particles, which affects the time for the particles to completely dissolve.

U.S. Pat. Nos. 6,017,559 and 6,074,986 teach the use of templating agents to control particle size in pesticide formulations. However, neither the '559 patent nor the '986 patent addresses the concerns with bioavailability of drug substances.

It would be an advantage to provide stable pharmaceutical compositions in the micron or submicron particle size range which have improved bioavailability but do not have the problems associated with the above identified prior art.

### SUMMARY OF THE INVENTION

In a first aspect, the present invention is a method for producing a micron-sized or submicron-sized drug which comprises preparing a template emulsion comprising water and a templating agent; preparing a drug-containing mixture comprising a drug substance; and combining the template emulsion with the drug-containing mixture to form a template emulsion loaded with drug particles.

The template emulsion loaded with drug particles may be used as is or if a solid dispersion of the drug substance is desired, the solvent may be removed from the template emulsion.

In a second aspect, the present invention is drug particles produced by a method which comprises preparing a template emulsion comprising water and a templating agent; preparing a drug containing mixture comprising a drug substance; and combining the template emulsion with the drug containing mixture to form drug particles in the template emulsion.

The present invention has several advantages. The present invention only imparts high temperature and mechanical stress, i.e., high shear, necessary to form small droplets, to the template emulsion and not to the drug containing mixture, thereby reducing the potential harm to the bioactivity of the drug substance. Moreover, no additional emulsifiers, other than those required to form the template emulsion, are required, thereby reducing the level of excipients present in the final drug formulation. In addition, the use of a templating agent provides improved control over the particle size and size distribution of the resulting drug particles.

### BRIEF DESCRIPTION OF THE FIGURES

FIG. 1 is a graph depicting the X-ray powder diffraction pattern of an embodiment of the present invention.

FIG. 2 is a graph depicting the in vitro dissolution profile of an embodiment of the present invention.

FIGS. 3A-3C are graphs depicting the in vivo bioavailability of an embodiment of the present invention.

### DETAILED DESCRIPTION OF THE INVENTION

The template emulsions used in the present invention are defined as being a stable two-phase dispersion comprising a continuous aqueous phase and a discontinuous phase comprising a non-aqueous material and a stabilizer in an amount sufficient to depress migration of the non-aqueous material through the aqueous phase, thereby diminishing or preventing particle growth of the template emulsion, for example through Ostwald ripening, wherein the stabilizer is soluble in the discontinuous phase but insoluble in the aqueous phase.

The template emulsions used to prepare the micron-sized and submicron-sized drug particles of the present invention are prepared using techniques well known in the art for forming emulsions and comprise a templating agent and water in an amount of from 0.5 to 50, preferably from 2 to 20, and most preferably from 5 to 10 percent by weight.

It is important that the template emulsion droplets have the proper size. As the drug containing mixture is combined with the template emulsion, the drug substance and solvent will migrate into the templating agent droplets, increasing the droplet size by a consistent and predictable amount. The size of the droplets of the templating agent in the template emulsion thus determines the size of the resulting drug particles by setting a boundary for the particle growth of the resulting drug particles.

By this method, the particle size distribution of the resulting drug particles in the template emulsion can be controlled by appropriate control of the droplet size distribution of the templating agent in the template emulsion. The shape of the particle size distribution curve of the resulting aqueous emulsion reflects closely the shape of the particle size distribution curve of the templating agent employed.

In order to form the appropriate droplet size for the templating agent in the template emulsion, some form of agitation is preferably used. The type of agitation used is not critical, and any type of conventional agitation used to make emulsions can be used, so long as the appropriate droplet size for the templating agent in the template emulsion is achieved. Examples of suitable agitation means include stirring, homogenization, and the use of a microfluidizer, micromixer etc.

Preferably, the templating agent is a liquid or oil that is poorly water soluble. Examples of suitable templating agents include alkyl substituted benzenes such as toluene, xylene or propyl benzene fractions, and mixed naphthalene and alkyl naphthalene fractions; mineral oils; triglyceride oils such as cottonseed oil, olive oil, soybean oil and vegetable oils; hydrated vegetable oils; dialkyl amides of fatty acids, particularly the dimethyl amides of fatty acids; chlorinated aliphatic and aromatic hydrocarbons such as 1,1,1,-trichloroethane and chlorobenzene; esters of glycol derivatives such as the acetate of the n-butyl, ethyl, or methyl ether of diethyleneglycol; ketones such as isophorone and trimethylcyclohexanone; and alkyl acetates such as hexyl or heptyl acetate. The preferred templating agents are those that are either listed by the FDA as generally regarded as safe (GRAS) or easily removable by standard procedures, i.e., cottonseed oil, olive oil, soybean oil and vegetable oils; mineral oils; alkyl acetates; and toluene.

In a preferred embodiment, the template emulsion further comprises at least one stabilizer. The stabilizer has several functions. The stabilizer operates as an emulsifying agent depressing the migration of the non-aqueous material through the aqueous phase, thereby stabilizing the template emulsion by diminishing or preventing droplet growth of the template emulsion. The stabilizer also inhibits crystal growth, aggregation and agglomeration of the drug particles. Examples of suitable stabilizers may be polymers, homopolymers or co-polymers, for example those described in “Polymer Handbook” 3^{rd} Edition edited by J. Brandrup and E. H. Immergut. Examples of suitable homopolymers and co-polymers include polyolefins and substituted polyolefins such as polyethylene, polypropylene, polybutene, polybutadiene, and chlorinated derivatives thereof; polyacrylates and polymethacrylates; polydisubstituted esters; polyvinyl ethers, chlorides, acetates, and carboxylate esters such as polyvinyl butyrate caprylate, laurate, stearate, benzoate; polystyrene; natural rubber and hydrochlorinated rubber; ethyl, butyl, and benzyl celluloses; cellulose esters; and combinations of these polymers. Other suitable polymers are those polymers which can also function as a surfactant but yet are insoluble in the continuous aqueous phase, such as nonionic polyalkylene glycol/(poly)carboxylic acid compounds; A-B-A block-type surfactants; and high molecular weight esters of natural vegetable oils such as the alkyl esters of stearic and oleic acids. In addition to polymers, very hydrophobic small molecules, i.e., hexadecane, can be employed as well. Preferred stabilizer are those that are a part of the GRAS-list, i.e., alkyl esters of stearic and oleic acids. Depending on the molecular weight or the degree of crosslinking, the stabilizer can be in the physical state of a liquid or oil, or can be a solid. Generally, the composition of the stabilizer or mixture of stabilizers will depend upon the need to exclusively interact with the dispersed phase but not interact with the continuous phase. The stabilizers may be employed in an amount from 0.1 to 90, preferably from 0.5 to 50 percent by weight of the dispersed phase.

In one embodiment, the stabilizer is a surfactant. Surfactants that can be advantageously employed herein can be readily determined by those skilled in the art and include various nonionic, anionic, cationic, and amphoteric surfactants, or a blend of those surfactants. Preferred surfactants are those which significantly reduce the tendency for the oil droplets of the discontinuous phase to agglomerate. Examples of nonionic surfactants include the polyalkylene glycol ethers and condensation products of aliphatic alcohols, aliphatic amines, or fatty acids with ethylene oxide or propylene oxide; polyvinyl alcohols of different molecular weights and degree of hydrolyzation; polyvinyl pyrrolidones; and the surfactants of the Brij, Tween, and Span series. Anionic surfactants include salts of alkyl aryl sulphonic acids, sulphated polyglycol ethers, and ethers of sulphosuccinic acid. Cationic surfactants include quaternary ammonium compounds and fatty amines. The surfactant is generally employed in an amount of from 0.1 to 15, more preferably from 2 to 10, and most preferably about 5 percent by weight of the total composition.

The template emulsion is combined with a drug containing mixture which is defined herein as being a drug solution or a coarse drug emulsion. A drug solution comprises a drug substance and a water immiscible solvent, whereas a coarse drug emulsion comprises a drug substance, a water immiscible solvent, and water.

Mixing of the template emulsion and the drug containing mixture is preferably carried out at a temperature of from ambient to 70° C., more preferably ambient to 50° C., and most preferably at ambient temperature. The appropriate temperature chosen will depend upon the melting points of the materials used in the preparation and the temperature stability of the drug. The template emulsion and the drug containing mixture can be combined using any technique known in the art of combining liquid streams. Some form of agitation is preferably applied, although the process does not require agitation to successfully load the drug substance into the template droplets. The type of agitation used is not critical, and any type of conventional agitation can be used. The drug substance is generally employed in an amount of from 1 to 50, more preferably from 15 to 30 percent by weight of the drug containing mixture used to load the template droplets with the drug. The appropriate drug-to-solvent ratio mostly depends on the solubility of the drug in the chosen solvent.

Preferably, the drug substance is in essentially pure form. The drug substance is preferably poorly soluble in water with a solubility range of between 0.1 and 10 percent by weight, and dispersible in at least one liquid medium. Preferred drug substances include those intended for oral administration including, for example, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiacinotropic agents, contrast media, corticosterioids, cough suppressants (expectorants and mucolytics), diagnostic agents, diagnostic imaging agents, diuretics, dopaminergics (antiparkinsonian agents), haemostatics, immuriological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasidilators and xanthines. A description of these classes of drugs and a listing of species within each class can be found in Martindale, The Extra Pharmacopoeia, Twenty-ninth Edition, The Pharmaceutical Press, London, 1989, the disclosure of which is hereby incorporate by reference.

The drug containing mixture utilizes at least one solvent such that when the drug containing mixture and the template emulsion are combined, the solvent forces the drug to migrate into the templating agent. Solvents preferred for use in the drug containing mixture must have low water solubility, preferably between 0.01 and 2.0 percent by weight, and low vapor pressure, preferably between 0.5 and 500 mm Hg. Suitable solvents include alkanes and chlorinated alkanes such as dichloromethane, aliphatic and aromatic ethers, alipatic and aromatic esters, such as IBA, aliphatic and aromatic ketones, aromatics such as toluene, and combinations thereof.

Once the template emulsion and the drug containing mixture are combined, the drug substance will migrate into the templating agent droplets, forming drug particles in the template emulsion. The size of such drug particles are in the submicron to micron range, between 0.2 and 20, more preferably between 0.5 and 10, and most preferably between 0.5 and 5 microns, as measured using light scattering techniques.

In a preferred embodiment, the process of the present invention further comprises the step of removing the solvents. The vast amount of the solvents can be removed from the template emulsion by evaporation using standard evaporation techniques, causing the drug substance to precipitate or crystallize. The drug particle size is hereby controlled by the size of the template emulsion droplets.

In another preferred embodiment, the process of the present invention comprises an additional solvent removal step and in particular, a water removal step. The final solvent removal can be done using any technique known in the art of drying, i.e., freeze-drying, spray-drying, fixed or fluidized bed drying, or flash drying; or the solid drug substance particles can be isolated from the aqueous phase by standard separation techniques.

The compositions of the inventions may also include optional excipients such as standard fillers, binders, or disintegrants readily known by those skilled in the art in amounts of 0 to 15 percent by weight of the total composition.

The resulting drug particles are desirably redispersible in water with nearly the same particle size as the particles before redispersion. Preferably, the particles are redispersed in water such that the resulting redispersed particle size is less than 5 microns.

The invention will be further clarified by a consideration of the following examples, which are intended to be purely exemplary of the present invention. All parts and percentages are by weight, unless otherwise specified.

### EXAMPLES

### Example 1

A template emulsion comprising cottonseed oil (2.5 g), polyvinylpyrrolidone 55 kD (7.5 g), methyloleate (1.1 g), and water (12.5 g) was prepared with high shear mixing. A fraction (4 g) of the template emulsion was diluted with water (32 g), giving template emulsion droplets with a mean diameter of 0.19 microns as measured by light scattering. The template emulsion was mixed at ambient temperature with a solution comprising the drug danazol (0.5 g) and the solvent dichloromethane (4.5 g). The particle mean diameter initially increased to 2.4 microns, and decreased within the next 20 hours to 0.35 microns after the drug solution was absorbed by the templating agent. The organic solvent was stripped from the emulsion by evaporation and the continuous phase removed by freeze-drying, producing a white crystalline solid. The white crystals were redispersed in water, initially giving particles with a mean diameter of 6.5 microns, which disintegrated within two hours to produce particles with a mean diameter of 0.3 microns.

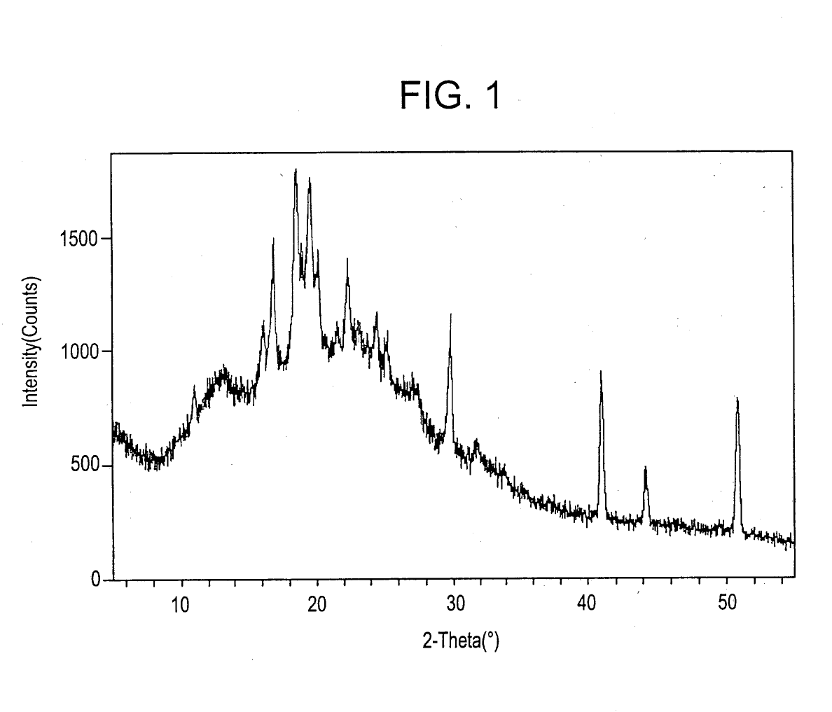

The crystallinity of the danazol particles was verified by X-ray powder diffraction (FIG. 1). The peak pattern of the danazol sample is essentially identical to the peak pattern of an untreated danazol control.

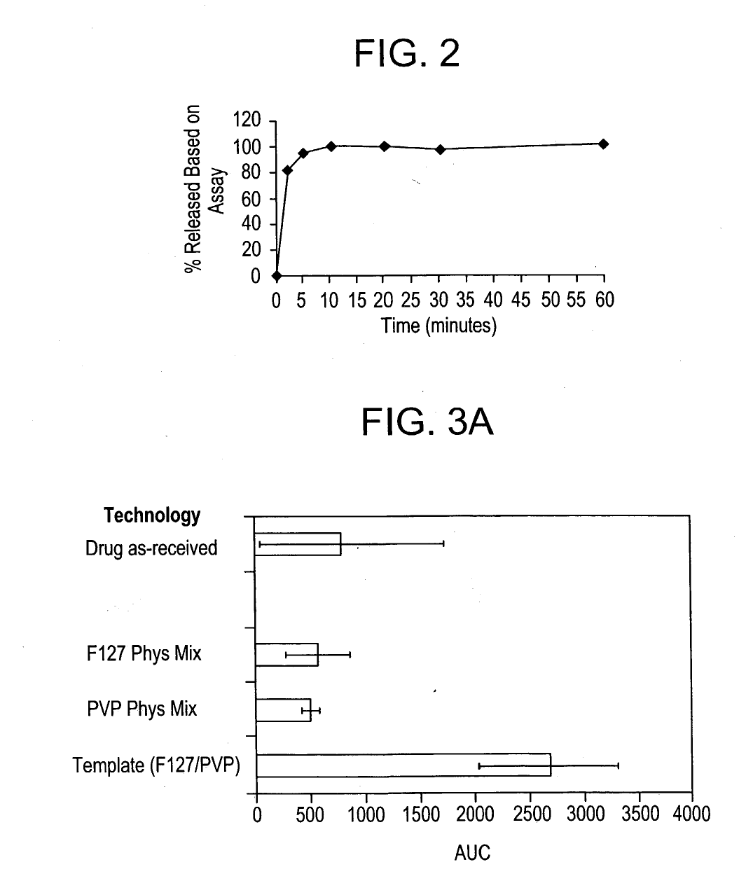

The high in-vitro dissolution rate of the danazol particles was verified by a standard dissolution protocol according to the USP 24 monographs (FIG. 2). Essentially all danazol dissolved within the first ten minutes.

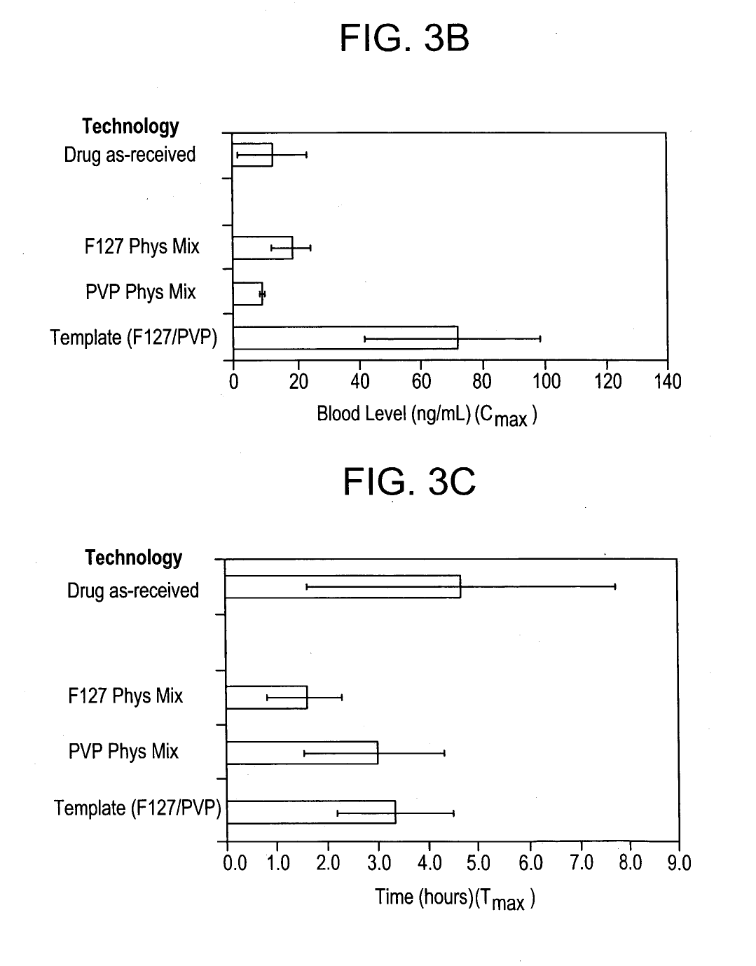

The improved bioavailability of the danazol particles was verified in an in-vivo study using rats who received 17.0 to 17.2 mg of danazol in a single oral gavage of capsules (FIGS. 3A-3C). The area under the curve (AUC) of the danazol template emulsion sample, shown in FIG. 3A, is clearly higher than the AUC's of the controls, i.e., danazol as received and physical mixtures of the drug danazol with the stabilizers Pluronic F-127 and polyvinylpyrrolidone 55 kD. The maximal blood level concentration (Cmax) of the danazol template emulsion sample, shown in FIG. 3B, is significantly higher than the maximal blood level concentrations measured for the controls, and the time to reach this maximal blood level concentration (Tmax), shown in FIG. 3C, is comparable to those of the controls. The results clearly demonstrate an enhanced bioavailability of the danazol template emulsion sample.

### Example 2

A template emulsion comprising triolein (7.5 g), polyvinylalcohol 9-10 kD (1.5 g), Span 80 (1.1 g), and water (7.5 g) was prepared with high shear mixing, giving template emulsion droplets with a mean diameter of 0.14 microns as measured by light scattering. The template emulsion was mixed at ambient temperature with a solution comprising the drug danazol (0.5 g) and the solvent dichloromethane (4.5 g). The particle mean diameter initially increased to 3.9 microns, and decreased within the next 20 hours to 0.4 microns after the drug solution was absorbed by the templating agent. The organic solvent was stripped from the emulsion by evaporation and the continuous phase removed by freeze-drying, producing a white crystalline solid. The white crystals were redispersed in water, initially giving particles with a mean diameter of 13.4 microns, which disintegrated within twelve hours to produce particles with a mean diameter of 0.8 microns.

### Example 3

A template emulsion comprising cottonseed oil (7.5 g), polyvinylalcohol 9-10 kD (7.5 g), and water (7.5 g) comprising 10 percent by weight Pluronic F-68 was prepared with high shear mixing, giving template emulsion droplets with a mean diameter of 0.3 microns as measured by light scattering. The template emulsion was mixed at ambient temperature with a solution comprising the drug cyclosporin A (0.5 g) and the solvent toluene (2.2 g). The particle mean diameter increased to 0.4 microns after the drug solution was absorbed by the templating agent and remained stable over at least 24 hours. The organic solvent was stripped from the emulsion by evaporation and the continuous phase removed by freeze-drying, producing a white powder sample. The white powder was redispersed in water, initially giving particles with a mean diameter of 14.5 microns, which disintegrated within five hours to produce particles with a mean diameter of 0.8 microns.

### Example 4

An aqueous template emulsion (5% in water) comprising stabilizer Atlox 4991 and methyloleate (12.5%) was prepared with high shear mixing. The template emulsion was mixed at ambient temperature with a coarse drug emulsion comprising the drug cyclosporin A (10%), the solvent toluene (40%), the stabilizer Tween 40 (5%), and water (45%). The initial particle mean diameter was 12.7 microns as measured by light scattering, but decreased to 3.6 microns after 15 minutes, 1.2 microns after 25 minutes, and 0.64 microns after 20 hours, while the drug emulsion was absorbed by the templating agent.

### Examples 5 and 6

A template emulsion (35 g) comprising cottonseed oil (0.89%), polyvinylpyrrolidone 55 kD (2.68%), methyloleate (0.39%), and water (96.04%) was prepared with high shear mixing (10 minutes at 20,000 rpm) at ambient temperature, giving template emulsion droplets with a volume mean diameter of 0.28 microns as measured by light scattering. The template emulsion was mixed at ambient temperature with a solution comprising the drug nifedipine (1 g) and the solvent toluene/isobutylacetate in a 60:40 mixing ratio (30 g), and the drug ketoconazole (1 g) and the solvent dichloromethane (9 g), respectively, as indicated in Table A. The organic solvent was stripped from the emulsion by evaporation and the continuous phase removed by freeze-drying, producing a dry powder sample. The powder sample was redispersed in deionized water. The volume mean diameter of the oil droplets measured after addition of the drug solution, and the volume mean diameter of the drug particles after drying and redispersion in water are listed in Table A.

For each example, the degree of crystallinity was determined using X-ray diffraction as known by those skilled in the art of particle size measurement, with aluminum oxide as the internal standard. Table A below lists the materials used and the results. As used in Table A, “PSA” means particle size analysis. “PSA redisp.” refers to the particle size of the redispersed particles.

TABLE A

| | | | PSA | PSA | % |
|---|---|---|---|---|---|
| Ex. | Drug | Organic Solvent | [μm] | redisp.[μm] | Crystal. |
| 5 | Nifedipine | Tol/IBA 60/40 | 1.208 | 2.210 | 80 |
| | | (30 g) | | | |
| 6 | Ketoconazole | dichloromethane | 0.507 | 3.200 | 65 |
| | | (9 g) | | | |

Tol/IBA 60/40 means toluene/isobutylacetate in a 60:40 mixing ratio.

### Examples 7 through 10

A template emulsion (13 g) comprising toluene (5%), polyvinylalcohol 9-10 kD (5%), Span 60 (0.37%), and water (89.6%) was prepared with high shear mixing (3 minutes at 20,000 rpm) at ambient temperature, giving template emulsion droplets with a volume mean diameter of 0.40 microns as measured by light scattering. The template emulsion was mixed at ambient temperature with a solution comprising the drug naproxen, cyclosporin A, nifedipine, or ketoconazole (0.5 g), respectively, and the solvents dichloromethane (4.5 g) or toluene/isobutylacetate 60:40, as indicated in Table B. The organic solvent was stripped from the emulsion by evaporation and the continuous phase removed by freeze-drying, producing a dry powder sample. The powder sample was redispersed in deionized water. The volume mean diameter of the oil droplets measured after addition of the drug solution, and the volume mean diameter of the drug particles after redispersion in water are listed in Table B.

For each example, the degree of crystallinity was determined using X-ray diffraction as known by those skilled in the art of particle size measurement, with aluminum oxide as the internal standard. Table B below lists the materials used and the results. As used in Table B, “PSA” means particle size analysis. “PSA redisp.” refers to the particle size of the redispersed particles.

TABLE B

| | | Organic | PSA | PSA | % |
|---|---|---|---|---|---|
| Ex. | Drug | Solvent | [μm] | redisp.[μm] | Crystal. |
| 7 | Naproxen | Tol/IBA | 0.385 | 1.194 | 100 |
| | | 60/40 (10 g) | | | |
| 8 | Cyclosporin A | Tol/IBA | 0.439 | 0.901 | amorphous |
| | | 60/40 (10 g) | | | |
| 9 | Nifedipine | Tol/IBA | 0.759 | 3.988 | 100 |
| | | 60/40 (15 g) | | | |
| 10 | Ketoconazole | dichloro- | 0.400 | 2.487 | amorphous |
| | | methane (4.5 g) | | | |

Tol/IBA 60/40 means toluene/isobutylacetate in a 60:40 mixing ratio.

The improved in-vitro dissolution rates of the template-prepared samples given in Examples 7 through 10 compared to “drug as received” controls were verified by standard dissolution protocols according to the USP 24 monographs. The dissolution progress was measured after 5, 10, 15, and 60 minutes using either High Pressure Liquid Chromatography (HPLC) or UV/VIS detection as the analytical tools. The results are listed in Table C as time in minutes versus percent of dissolved sample for the template-prepared samples. For comparison, the corresponding data for the controls are given in parenthesis. All template formulations show an enhanced dissolution rate, especially during the first 15 minutes, which are crucial for a fast uptake of the drugs.

TABLE C

| Ex. | Drug | 5 min | 10 min | 15 min | 60 min |
|---|---|---|---|---|---|
| 7 | Naproxen | 48% (7%) | 55% (11%) | 59% (15%) | 80% |
| | | | | | (37%) |
| 8 | Cyclosporin A | 15% (15%) | 34% (23%) | 56% (29%) | 100% |
| | | | | | (87%) |
| 9 | Nifedipine | 61% (43%) | 80% (44%) | 89% (45%) | 97% |
| | | | | | (54%) |
| 10 | Ketoconazole | 50% (31%) | 68% (41%) | 78% (48%) | 100% |
| | | | | | (100%) |

What is claimed is:

1. A method of producing a micron-sized or submicron-sized drug which comprises:
(a) preparing a template emulsion comprising water and a templating agent;
(b) preparing a drug-containing mixture comprising a drug substance; and
(c) combining the template emulsion with the drug-containing mixture to form drug particles in the template emulsion.

2. The method according to claim 1, wherein the template emulsion further comprises at least one stabilizer.

3. The method according to claim 1, wherein the drug-containing mixture further comprises at least one solvent.

4. The method according to claim 3 wherein the solvent is poorly soluble in water.

5. The method according to claim 3 wherein the solvent has low vapor pressure.

6. The method according to claim 3 wherein the method further comprises the step of removing the solvent.

7. The method according to claim 6 wherein the method further comprises the step of removing water.

8. The method according to claim 1 wherein the average particle size of the drug particles in the template emulsion is from 0.25 microns to 15 microns.

9. The method according to claim 1, wherein the templating agent is poorly soluble in water.

10. The method according to claim 9, wherein the templating agent is selected from the group consisting of alkyl substituted benzenes, cottonseed oil, olive oil, soybean oil, vegetable oils, hydrated vegetable oils, dialkyl amides of fatty acids, chlorinated aliphatic hydrocarbons, chlorinated aromatic hydrocarbons, esters of glycol derivatives, ketones, mineral oils, alkyl acetates and toluene.

11. The method according to claim 2, wherein the stabilizer is selected from the group consisting of polymers, homopolymers, co-polymers, surfactants and combinations thereof.

12. The method according to claim 3, wherein the solvent is selected from alkanes, chlorinated alkanes, aliphatic ethers, aromatic ethers, alipatic esters, aromatic esters, alipatic ketones, aromatic ketones and combinations thereof.

13. Drug particles produced by a method which comprises:
(a) preparing a template emulsion comprising water and a templating agent;
(b) preparing a drug containing mixture comprising a drug substance; and
(c) combining the template emulsion with the drug containing mixture to form drug particles in the template emulsion.

14. Drug particles according to claim 13, wherein the template emulsion further comprises at least one stabilizer.

15. Drug particles according to claim 13, wherein the drug containing mixture further comprises at least one solvent.

16. Drug particles according to claim 15 wherein the method further comprises the step of removing the solvent.

17. Drug particles according to claim 15 wherein the solvent is poorly soluble in water.

18. Drug particles according to claim 15 wherein the solvent has low vapor pressure.

19. Drug particles according to claim 13 wherein the average particle size of the drug in the template emulsion is from 0.25 microns to 15 microns.

20. Drug particles according to claim 13, wherein the templating agent is poorly soluble in water.

21. Drug particles according to claim 20, wherein the templating agent is selected from the group consisting of alkyl substituted benzenes, cottonseed oil, olive oil, soybean oil, vegetable oils, hydrated vegetable oils, dialkyl amides of fatty acids, chlorinated aliphatic hydrocarbons, chlorinated aromatic hydrocarbons, esters of glycol derivatives, ketones, mineral oils, alkyl acetates and toluene.

22. Drug particles according to claim 14 wherein the stabilizer is selected from the group consisting of polymers, homopolymers, co-polymers, surfactants and combinations thereof.

23. Drug particles according to claim 15, wherein the solvent is selected from alkanes, chlorinated alkanes, aliphatic ethers, aromatic ethers, alipatic esters, aromatic esters, alipatic ketones, aromatic ketones and combinations thereof.
